(19) United States
(12) Patent Application Publication
(10) Pub. No.: US 2007/0081969 A1
(43) Pub. Date: Apr. 12, 2007

(54) Dissemination apparatus

(75) Inventors: Thomas McGee, Nyack, NY (US); Richard P. Sgaramella, Hoboken, NJ (US)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 10/577,089

(22) PCT Filed: Oct. 28, 2004
(86) PCT No.: PCT/CH04/00647

§ 371 Date: Jun. 27, 2006

Related U.S. Application Data
(60) Provisional application No. 60/518,842, filed on Nov. 10, 2003.

(51) Int. Cl. A61L 9/00; F26B 13/10

(52) U.S. Cl. 424/76.2; 34/523

(57) ABSTRACT

An apparatus for disseminating volatile liquid such as fragrance or insecticide into an atmosphere from a reservoir, the transfer to atmosphere being at least partially achieved by means of a transfer member having external capillary channels. The volatile liquid is one in which at least 30% by weight of the materials therein have a molecular weight of 175 maximum, and which has a surface tension of less than 40 dynes/cm. The transfer member is of plastics material having a surface energy of less than 45 dyne/cm. The combination allows for particularly efficient dissemination.

This invention relates to apparatus for the disseminating of volatile liquids into an atmosphere.

One very common method apparatus for disseminating a volatile liquid, such as a fragrance or an insecticide, into an atmosphere consists of a porous transfer member, such as a porous wick, that is in contact with a reservoir of volatile liquid. Liquid rises up this wick and evaporates into the atmosphere. This system has drawbacks, such as the low surface area for evaporation and the tendency for the wick to fractionate complex mixtures, such as fragrances, so that some components are disseminated earlier than others and the full effect of the fragrance is lost.

It has been proposed to overcome this disadvantage by using external capillaries, that is, capillary channels cut or moulded into a suitable substrate. One example is described in U.S. Pat. No. 4,913,350, in which an external capillary channel-containing member is inserted into a liquid. In another embodiment, described in United Kingdom Patent Application GB 0306449, there is fitted to a known transfer member a capillary sheet, that is, a sheet extending essentially perpendicularly from the transfer member and comprising channels of capillary dimensions, to which volatile liquid can pass and travel along for evaporation. This sheet generally contacts the transfer member by means of a hole in the sheet through which the transfer member protrudes and within which it fits snugly, at least some of these channels contacting the transfer member such that liquid can transfer from the member to the sheet (“liquid transfer contact”).

Although this technology offers significant advantages over the porous wicks of the art, these advantages have never been completely realized. It has now been found that it is possible to obtain the full benefits of the technology by adherence to certain fundamental parameters. The invention therefore provides an apparatus adapted to disseminate volatile liquid into an atmosphere from a reservoir, the transfer to atmosphere being at least partially achieved by means of a transfer member having external capillary channels, characterised in that
(a) at least 30% by weight of the materials comprising the volatile liquid have a molecular weight of 175 maximum and the volatile liquid has a surface tension of less than 40 dynes/cm; and
(b) The transfer member is of plastics material having a surface energy of less than 45 dyne/cm.

By “at least 30% by weight” is meant all the components of the liquid, including any solvent present.

When the active is a fragrance it can be composed with one or more compounds, for example, natural products such as extracts, essential oils, absolutes, resinoids, resins, concretes etc., but also synthetic materials such as hydrocarbons, alcohols, aldehydes, ketones, ethers, acids, esters, acetals, ketals, nitrites, etc., including saturated and unsaturated compounds, aliphatic, carbocyclic, and heterocyclic compounds. The molecular weights range from around 90 to 320. Such fragrance materials are mentioned, for example, in S. Arctander, Perfiume and Flavor Chemicals (Montclair, N.J., 1969), in S. Arctander, perfume and Flavor Materials of Natural Origin (Elizabeth, N.J., 1960) and in “Flavor and Fragrance Materials—1991”, Allured Publishing Co. Wheaton, Ill. USA.

Some non-limiting examples of useful volatile materials whose molecular weight is less than 175 are:

| | Molecular |
|---|---|
| Material | Weight |
| ethyl acetate | 88 |
| iso-amyl alcohol | 88 |
| 2-methylpyrazine | 94 |
| cis 3-hexenol | 100 |
| C6-aldehyde | 100 |
| C6 alcohol | 102 |
| ethyl propionate | 102 |
| benzaldehyde | 106 |
| benzyl alcohol | 108 |
| C7-aldehyde | 114 |
| methyl amyl ketone | 114 |
| iso amyl formate | 116 |
| ethyl butyrate | 116 |
| Indole | 117 |
| acetophenone | 120 |
| phenyl ethyl alcohol | 122 |
| styralyl alcohol | 122 |
| Veltol ™ | 126 |
| methyl hexyl ketone | 128 |
| 3-methyl 3-methoxy butanol | 128 |
| ethyl amyl ketone | 128 |
| octenol JD | 128 |
| prenyl acetate | 128 |
| C8-aldehyde | 128 |
| amyl acetate | 130 |
| cinnamic aldehyde | 132 |
| phenyl propyl aldehyde | 134 |
| cinnamic alcohol | 134 |
| terpinolene | 136 |
| phenyl acetic acid | 136 |
| phenyl propyl alcohol | 136 |
| alpha pinene | 136 |
| benzyl formate | 136 |
| anisic aldehyde | 136 |
| d-limonene | 136 |
| Triplal ™ | 138 |
| Cyclal C ™ | 138 |
| Melonal ™ | 140 |
| C-9 aldehyde | 142 |
| iso nonyl aldehyde | 142 |
| cyclo hexyl acetate | 142 |
| ethyl caproate | 144 |
| hexyl acetate | 144 |
| coumarin | 146 |
| methyl cinnamic aldehyde | 146 |
| cuminic aldehyde | 148 |
| benzyl acetone | 148 |
| geranyl nitrile | 149 |
| cuminyl alcohol | 150 |
| benzyl acetate | 150 |
| Heliotropine ™ | 150 |
| thymol | 150 |
| neral | 152 |
| synthetic vanillin | 152 |
| synthetic citral | 152 |
| rose oxide | 154 |
| geraniol | 154 |
| allyl caproate | 156 |
| Rosalva ™ | 156 |
| tetrahydro myrcenol | 158 |
| yara yara | 158 |
| diethyl malonate | 160 |
| methyl cinnamate | 162 |
| Jasmorange ™ | 162 |
| benzyl propionate | 164 |
| eugenol | 164 |
| ethyl vanillin | 166 |
| dihydrojasmone | 166 |
| geranic acid | 168 |
| methyl laitone | 168 |
| methyl nonyl ketone | 170 |
| methyl tuberate | 170 |
| hexyl butyrate | 172 |
| octyl-3-acetate | 172 |
| hydroxycitronellol | 174 |
| Fructone ™ | 174 |

Some non-limiting examples of useful materials that can be used that have a molecular weight higher than 175 are:

| | Molecular |
|---|---|
| Material | Weight |
| benzal glyceryl acetal | 180 |
| anisyl acetate | 180 |
| terpinyl formate | 182 |
| geranyl formate | 182 |
| methyl diphenyl ether | 184 |
| delta undecalactone | 184 |
| allyl amyl glycolate | 186 |
| amyl caproate | 186 |
| Fraistone ™ | 188 |
| Pelargene ™ | 188 |
| Florhydral ™ | 190 |
| ethyl hexyl ketone | 190 |
| ethyl phenyl glycidate | 192 |
| Verdyl acetate ™ | 192 |
| dihydro beta ionone | 194 |
| iso-butyl salicylate | 194 |
| allyl cyclo hexyl propionate | 196 |
| myrcenyl acetate | 196 |
| citronellyl oxyacetaldehyde | 198 |
| citral dimethyl acetal | 198 |
| beta naphthyl iso butyl ether | 200 |
| tetrahydro linalyl acetate | 200 |
| amyl cinnamic aldehyde | 202 |
| Fruitaflor ™ | 202 |
| Lilial ™ | 204 |
| damascenone | 204 |
| methyl ionone | 206 |
| Cashmeran ™ | 206 |
| Ebanol ™ | 206 |
| phenoxy ethyl iso butyrate | 208 |
| iso amyl salicylate | 208 |
| Sandalore ™ | 210 |
| propyl diantilis | 210 |
| benzyl benzoate | 212 |
| citronellyl propionate | 212 |
| myristic alcohol | 214 |
| Gelsone ™ | 214 |
| hexyl cinnamic aldehyde | 216 |
| butyl butyryllactate | 216 |
| amyl cinnamate | 218 |
| hydroxycitronellal dimethyl acetal | 218 |
| beta methyl ional | 220 |
| Vetiverol ™ | 220 |
| hexyl salicylate | 222 |
| geranyl crotonate | 222 |
| methyl jasmonate | 224 |
| linalyl butyrate | 224 |
| Hedione ™ | 226 |
| Timberol ™ | 226 |
| Floramat ™ | 228 |
| benzyl salicylate | 228 |
| Fixal ™ | 230 |
| Cetone V ™ | 232 |
| cis carveol | 232 |
| Iso E Super ™ | 234 |
| muscalone | 234 |
| geranyl tiglate | 236 |
| Cetalox ™ | 236 |
| linalyl valerate | 238 |
| benzyl cinnamate | 238 |
| Thibetolide ™ | 240 |
| phenyl ethyl phenylacetate | 240 |
| phenyl ethyl salicylate | 242 |
| Boisambrene ™ | 242 |
| jasmonyl | 244 |
| Phantolid ™ | 244 |
| methyl cedryl ketone | 246 |
| Aldrone ™ | 248 |
| amyl cinnamic aldehyde dma | 248 |
| Dione ™ | 250 |
| cedryl formate | 250 |
| ambrettolide | 252 |
| phenyl ethyl cinnamate | 252 |
| benzyl iso eugenol | 254 |
| hexadecanolide | 254 |
| Novalide ™ | 256 |
| citronellyl ethoxalate | 256 |
| Fixolide ™ | 258 |
| Galaxolide ™ | 258 |
| rose acetate | 262 |
| ambrate | 262 |
| iso caryl acetate | 264 |
| cinnamyl cinnamate | 264 |
| ethyl undecylenate | 266 |
| Ethylene Brassylate ™ | 272 |
| triethyl citrate | 276 |
| dihexyl fumarate | 284 |
| Okoumal ™ | 288 |
| musk ketone | 294 |
| alpha Santalol ™ | 300 |
| geranyl iso valerate | 312 |

The solvent of the volatile liquid can be selected from many classes of volatile compounds that known to the art, for example, ethers; straight or branched chain alcohols and diols; volatile silicones; dipropylene glycol, triethyl citrate, ethanol, isopropanol, diethyleneglycol monoethyl ether, dipropylene glycol, diethyl phthalate, triethyl citrate, isopropyl myristate, etc., hydrocarbon solvents such as Isopar™ or other known solvents that have previously been used to dispense volatile actives from substrates. These solvents in general have a molecular weight between 20 and 400. They are selected specifically for each volatile liquid to achieve the performance and safety, (e.g. VOC and flash point) specified.

When the active is an insect repellant it can be composed of one or more compounds such as pyrethrum and pyrethroid type materials commonly now used in mosquito coils are likely to be the most useful for this purpose. Other insect control actives can be used, such as the repellants DEET, essential oils, such as citronella, lemon grass oil, lavender oil, cinnamon oil, neem oil, clove oil, sandalwood oil and geraniol.

When the active is an antimicrobial it can be composed of one or more of compounds such as essential oils such as rosemary, thyme, lavender, eugenic, geranium, tea tree, clove, lemon grass, peppermint, or their active components such as anethole, thymol, eucalyptol, farnesol, menthol, limonene, methyl salicylate, salicylic acid, terpineol, nerolidol, geraniol, and mixtures thereof. benzyl alcohol, ethylene glycol phenyl ether, propylene glycol phenyl ether, propylene carbonate, phenoxyethanol, dimethyl malonate, dimethyl succinate, diethyl succinate, dibutyl succinate, dimethyl glutarate, diethyl glutarate, dibutyl glutarate, dimethyl adipate, diethyl adipate, dibutyl adipate, or mixtures thereof one or more aldehydes selected from cinnamic aldehyde, benzaldehyde, phenyl acetaldehyde, heptylaldehyde, octylaldehyde, decylaldehyde, undecylaldehyde, undecylenic aldehyde, dodecylaldehyde, tridecylaldehyde, methylnonyl aldehyde, didecylaldehyde, anisaldehyde, citronellal, citronellyloxyaldehyde, cyclamen aldehyde, alpha-hexyl cinnamic aldehyde, hydroxycitronellal, alpha-methyl cinnamic aldehyde, methylnonyl acetaldehyde, propylphenyl aldehyde, citral, perilla aldehyde, tolylaldehyde, tolylacetaldehyde, cuminaldehyde, Lilial™, salicyl aldehyde, alpha-amylcinnamic aldehyde and Heliotropine™.

Other volatile actives can be used alone or in combination with the above actives, for example decongestants such as menthol, camphor, eucalyptus etc., malodor counteractants such as are trinmethyl hexanal, other alkyl aldehydes, benzaldehyde, and vanillin, esters of alpha-, beta-unsaturated monocarboxylic acids, alkyl cyclohexyl alkyl ketones, derivatives of acetic and propionic acids, 4-cyclohexyl-4-methyl-2-pentanone, aromatic unsaturated carboxylic esters, etc.

Care must be taken when designing the volatile liquid in that they pose no danger to the public. This is done by ensuring that the said volatile liquid has a flashpoint greater than about 60° C. as determined by Test Method ASTM D93.

The transfer medium must have external capillary channels, that is, channels of capillary dimensions provided on an external surface of the medium such that a liquid will exhibit capillary flow within them. These may be provided by any suitable means, such as moulding and engraving. The transfer medium may be any suitable form of such medium, but is preferably one of two kinds:

1. The type in which a member bearing external capillary channels contacts directly a liquid in a reservoir, and the liquid rises in the capillary channels and evaporates into the atmosphere. An example of such a type is described in U.S. Pat. No. 4,193,350

2. A type in which the liquid in the reservoir is taken therefrom by a porous wick in contact with it, there being mounted on the wick a capillary sheet whose external capillary channels are in liquid transfer contact with the wick, the liquid passing from the wick to the capillary channels and evaporating into the atmosphere. An example of such an apparatus is described in UK patent application GB 0306449

For the working of this invention, it is essential that the volatile liquid have a surface tension of 40 dynes/cm maximum and that the plastics material have a surface energy of 45 dynes/cm maximum. It has been found that this combination of parameters allows for an especially good dissemination of a liquid into an atmosphere. The invention therefore also provides a method of disseminating a volatile liquid into an atmosphere by evaporation from a transfer member having surface capillary channels, the volatile liquid being such that at least 30% by weight of the materials comprising it have a molecular weight of 175 maximum, and that it has a surface tension of less than 40 dynes/cm, and the transfer member being of plastics material having a surface energy of less than 45 dyne/cm.

The provision of a volatile liquid having the abovementioned characteristics is well within the skill of the art.

Preferably the liquid has a surface tension of less than 40 dyne/cm, and is more preferably within the range 20-35 dynes/cm. All surface tensions referred to herein are measured on a Fisher Surface Tensiomat model number 21 at 25° C.

It is further preferred that the volatile liquid have a viscosity of less than 10 centistokes per second at 25° C. as measured on a Cannon-Fenske Viscometer according to Test Method ASTM D 445.

The plastics materials for use in this invention preferably have a surface energy of from 15-45 dyne/cm. The surface energy of a plastics material is dependent upon its molecular structure and is a measure of the ability of a surface to be wetted. The more inert is a plastics material chemically, the lower is its surface energy. Thus, materials such as polyethylene, polypropylene and PTFE have low surface energies, whereas the plastics with more polar groups have higher surface energies. Preferably the surface energy lies in the range of from 30-45 dynes/cm and more preferably from 30-35 dyne/cm. Some suitable materials for the purposes of this invention are shown in the following table:

| | Example | | Surface |
|---|---|---|---|
| | Material Trade | | Energy |
| Material Name | Name(s) | Supplier | Dynes/cm |
| Polytetrafluoro- | TEFLON | DU PONT | 18 |
| ethylene PTFE | FEP106N | | |
| Polyethylene | BOREALIS MG | NORTHERN | 30 |
| PE (HDPE) | 9641-R | PLASTICS | |
| Polyethylene | IPETHENE 320 | CARMEL | 30 |
| PE (LDPE) | | OLEFINS | |
| Polyethylene | LL6201 | EXXON MOBIL | 30 |
| PE (LLDPE) | | | |
| Polystyrene PS | PS 146L | NOVA | 36 |
| | | CHEMICALS | |
| Polyvinylchloride | | | 41 |
| PVC | | | |
| Polyethylene | RADITER | RADICI | 42 |
| terepthalate PET | | (PLASTRIBUTION) | |
| Polycarbonate PC | LUPILON S- | MITSUBISHI | 40 |
| | 3000R | POLYMERS | |
| Polyvinyl- | EXP 058 | EXXON MOBIL | 32 |
| propylene PP | | | |

(TEFLON, BOREALIS, IPETHENE, RADITER and LUPILON are trade marks)

Suitable transfer members may be easily fabricated by known means, for example, by the methods described in the abovementioned U.S. 4,913,350 and GB application 0306449.

The invention is further described by the following non-limiting examples.

### EXAMPLE 1

Capillary sheets of polypropylene BP 400Ca 70, measuring 2.5 cm×7.5 cm and having a surface energy of 32 dyne/cm, were immersed to a depth of 1.25 cm. into 10 g of a number of vanilla fragrances containing different amounts of volatile materials with a MW less than 175. The quantity of fragrance diffused into the air was determined by weighing the container with fragrance and capillary. The following results were obtained after 4 days.

| Fragrance | % MW < 175 | Wt loss g/day |
|---|---|---|
| A1 | 14.5 | 0.35 |
| A2 | 34.5 | 0.87 |
| A3 | 53.6 | 0.64 |
| A4 | 61.6 | 0.69 |
| A5 | 69.05 | 1.10 |
| A6 | 75.6 | 0.84 |
| A7 | 81.6 | 0.86 |
| A8 | 93.5 | 0.97 |
| A9 | 93.5 | 1.07 |

This shows that, for effective transmission of fragrance into the atmosphere, the composition must have at least 30% of the fragrance materials with a molecular weight of less than 175.

### EXAMPLE 2

Two frusto-conical polyester wicks were placed in 11.5 g of A1 and A2 fragrances in Barex™ containers and allowed to equilibrate overnight. 1.5 mm thick polypropylene external capillary sheets with a central hole that allowed them to be fitted to the wicks were placed thereon, and the quantity of fragrance diffused per day was measured. The results after 6 days are shown below:

| Fragrance | % MW < 175 | Weight Loss g/day |
|---|---|---|
| A1 | 14.5 | 0.4 |
| A2 | 35.5 | 1.0 |

For a hybrid system i.e. one in which the transport of the fragrance is via a porous wick and the diffusion is via an external capillary, good diffusion is obtained when the fragrance has a quantity of components with a MW<175 is around 30% or higher

### EXAMPLE 3

Capillary sheets of polypropylene BP 400Ca 70, measuring 2.5cm×7.5 cm external capillary and having a surface energy of 32 dyne/cm, were immersed to a depth of 1.25cm into 10 g of a series of fragrances having more than 30% components with MW<175, but with different surface tensions. The surface tension was measured at 25° C. using a Fisher Surface Tensiomat model number 21.

The quantity of fragrance diffused into the air was determined by weighing the container with fragrance and capillary. The following results were obtained after 2 days:

| | | Surface tension |
|---|---|---|
| Fragrance | Wt Loss g/day | Dynes/cm |
| B1 | 1.1 | 35.6 |
| B2 | 0.7 | 38.2 |
| B3 | 0.5 | 41.2 |
| B4 | 0.5 | 42.2 |

This shows the advantage of having a surface tension below 40, and preferably below 38, dynes/cm.

### EXAMPLE 4

A capillary sheet of polypropylene BP 400Ca 70, measuring 2.5cm×7.5 cm and having a surface energy of 32 dyne/cm, was immersed to a depth of 1.25 cm into 10 g of a series of fragrances having more than 30% components with MW<175, but with different viscosities, The viscosity was measured using a Cannon-Fenske Viscometer by ASTM D 445.

The quantity of fragrance diffused into the air was determined by weighing the container with fragrance and capillary. The following results were obtained after 2 days:

| | | Viscosity |
|---|---|---|
| Fragrance | Wt Loss g/day | Cs/s |
| C1 | 0.4 | 13.7 |
| C2 | 0.4 | 11.9 |
| C3 | 0.4 | 10.6 |
| C4 | 0.9 | 8.2 |
| C5 | 1.1 | 6.0 |

For good diffusion, the viscosity of the fragrance should be below 10 Cs/s.

### EXAMPLE 5

Capillary sheets with different surface energies were set up as per example 1 with fragrance D (% Components MW<175>30, surface tension 37 dynes/cm and viscosity 5.7 Cs/s) and fragrance E (% Components MW<175>30, Viscosity 2.9 cS/s and surface tension 34.5 dynes/sec), respectively. The fragrances had an oil-soluble dye added and the height to which it rose (as a percentage of the height of the capillary) after 6 minutes was measured and recorded, and is shown in the following tables.

TABLE 5
Effect of surface energy on diffusion of fragrance D

| | Surface | |
|---|---|---|
| | energy | |
| Plastic | dynes/cm | Rise 6 min |
| PP BP 400 | 32 | 100(3) |
| PETG | 41 | 81 |
| PB ABS | 46 | 59 |

The 100% rise in PP BP 400 was achieved after only 3 minutes.

TABLE 6
Effect of surface energy on diffusion of fragrance E.

| | Surface | |
|---|---|---|
| | energy | |
| Plastic | dyne/cm | Rise 6 min |
| PP BP 400 | 32 | 100(1.2) |
| PETG | 41 | 100(2) |
| PB ABS | 46 | 41 |

100% rise was found after 1.2 min and 2 min, respectively for PP BP 400 and PETG.

This shows that the surface energy of the plastics material of the external capillary should be below 45 dynes/cm, preferably below 40 dynes/cm.

1. An apparatus adapted to disseminate volatile liquid into an atmosphere from a reservoir, the transfer to atmosphere being at least partially achieved by means of a transfer member having external capillary channels, characterised in that
(a) at least 30% by weight of the materials comprising the volatile liquid have a molecular weight of 175 maximum and the volatile liquid has a surface tension of less than 40 dynes/cm; and
(b) the transfer member is of plastics material having a surface energy of less than 45 dyne/cm.

2. An apparatus according to claim 1, in which the surface tension of the liquid is from 20-35 dynes/cm.

3. An apparatus according to claim 1, in which the surface energy of the plastics material is from 15-45 dynes/cm.

4. An apparatus according to claim 3, in which the surface energy lies in the range of from 30-45 dynes/cm.

5. An apparatus according to claim 4, in which the surface energy lies in the range of from 30-35 dynes/cm.

6. An apparatus according to claim 1, in which the volatile liquid has a viscosity of less than 10 centistokes per second at 25° C.

7. An apparatus according to claim 1 in which the transfer member bears external capillary channels, which directly contact a liquid in a reservoir, and the liquid rises in the capillary channels and evaporates into the atmosphere.

8. An apparatus according to claim 1, in which the liquid in the reservoir is taken therefrom by a porous wick in contact with it, there being mounted on the wick a capillary sheet whose external capillary channels are in liquid transfer contact with the wick, the liquid passing from the wick to the capillary channels and evaporating into the atmosphere.

9. A method of disseminating a volatile liquid into an atmosphere by evaporation from a transfer member having surface capillary channels, the volatile liquid being such that at least 30% by weight of the materials comprising it have a molecular weight of 175 maximum, and that it has a surface tension of less than 40 dynes/cm, and the transfer member being of plastics material having a surface energy of less than 45 dyne/cm.
